(19) United States
(12) Patent Application Publication
(10) Pub. No.: US 2011/0295260 A1
(43) Pub. Date: Dec. 1, 2011

(54) KNEE RECONSTRUCTION PROCEDURE AND SURGICAL IMPLEMENT PARTICULARLY USEFUL FOR SUCH KNEE RECONSTRUCTION PROCEDURE

(75) Inventors: Ran Oren, Kibbutz Gaaton- Doar-Na Oshrat (IL); Lee Ranon, Nahariya (IL); Elad Rash, Moshav Bet-Lehem HaGlilit - Doar-Na HaAm (IL)

(73) Assignee: T.A.G. Medical Devices - Agriculture Cooperative Ltd., Doar-Na Oshrat (IL)

(21) Appl. No.: 13/150,330

(22) Filed: Jun. 1, 2011

Related U.S. Application Data
(63) Continuation-in-part of application No. 12/859,580, filed on Aug. 19, 2010.
(60) Provisional application No. 61/274,690, filed on Aug. 20, 2009.
(60) Provisional application No. 61/350,071, filed on Jun. 1, 2010.

(51) Int. Cl. A61B 17/16; A61B 17/56

(52) U.S. Cl. 606/80; 606/86 R

(57) ABSTRACT

A surgical implement for use in a surgical procedure involving the manipulation of a wire or like article in a body cavity. The surgical implement includes: an elongated stem; a handle carried at the proximal end of the elongated stem; a socket in the form of a removable insert, carried at the distal end of the elongated stem and configured for insertion into a body cavity during the surgical procedure and for receiving the wire or like article; an actuator carried by the elongated stem and movable from a normal position closing the socket at the distal end of the elongated stem, to an actuated position opening the socket; a finger-piece carried by the actuator adjacent the handle so as to be conveniently engageable by a user, while gripping the handle, to move the actuator from its normal position closing the socket, to its actuated position opening the socket; and a spring urging the actuator to its normal position closing the socket. The described preferred embodiment is one wherein the surgical procedure is a knee reconstruction procedure, and the socket is configured to enter through a lateral-medial portal of the knee into the intercondylar notch of the femur bone for forming the femoral tunnel therein.

### RELATED APPLICATIONS

This application is a continuation-in-part (CIP) of pending U.S. patent application Ser. No. 12/859,580 filed Aug. 19, 2010, which claims the benefit of priority under 35 USC 119(e) of U.S. Provisional Patent Application No. 61/274,690 filed Aug. 20, 2009. This application also claims the benefit of priority under 35 USC 119(e) of U.S. Provisional Patent Application No. 61/350,071 filed Jun. 1, 2010. The contents of all of the above applications are incorporated by reference as if fully set forth herein.

### FIELD AND BACKGROUND OF THE INVENTION

The present invention relates to a method for performing an arthroscopic reconstruction surgical procedure involving the replacing of a cruciate ligament in the knee joint space connecting the femur bone to the tibia bone.

The invention also relates to a surgical implement for use in a surgical procedure involving the manipulation of a wire or like article in a body cavity. The invention is particularly useful for implements to be used in the novel arthroscopic knee reconstruction procedure, and is therefore described below with respect to such an application, but it will be appreciated that the implement could also be used in other procedures.

### BACKGROUND OF THE INVENTION

Several orthopedic surgical procedures performed with endoscopic (or arthroscopic) techniques require the creation of bone tunnels in a precise location and direction. In these procedures direct visual control of placing the tunnels is possible only partially. An example of such problems are those encountered in cruciate ligament reconstruction procedures.

Cruciate ligaments, which connect the thigh bone (femur) to the leg bone (tibia) crossways within the knee joint, are important for both the static and the dynamic stability of the joint. The two cruciate ligaments originate from the inner surface of the medial and the lateral femoral condyles and have insertion points located on the opposite side of the tibial plateau, forming an X-shape pattern. The Anterior Cruciate Ligament (ACL) originates from the lateral condyle and is frontal to the Posterior Cruciate Ligament (PCL). When these ligaments are damaged or torn, loss of stability results. Stability can be restored surgically by substituting a suitable graft as a replacement for the lost ligament. In order to restore normal function, it is important to position the graft to the natural origin and insertion locations.

Reconstructive surgery has been practiced for many years with considerable success, and the number of procedures performed has been growing steadily. As with many other surgical procedures, minimal invasive techniques have replaced open surgery, and most cruciate ligament reconstruction procedures are now performed arthroscopically. In most cases the graft chosen is autologous, i.e. taken from the patient's own body, derived either from the mid-section of the lower patellar tendon or from hamstring tendons.

In reconstructive surgery the knee is held at a 90° flexion and bone tunnels are formed in the femur and the tibia to receive the graft. Typically, the tibial tunnel is formed first. A Kirschner guide wire is inserted from the antero-medial surface of the tibia, under visualization, targeting the midpoint of the normal insertion on the tibial plateau—in case of ACL reconstruction on the medial side. When the guide-wire is in the correct position, it is over-drilled with a cannulated drill to form the tibial tunnel.

The midpoint of the origin of the ACL lies at 9 o'clock on the intercondylar notch. The femoral tunnel should ideally be drilled from the native origin location, at an angle suitable to provide sufficient length for fixation in the femur. However, this angle is offset from the centerline of the conventionally drilled tibial tunnel. Drilling the femoral tunnel as a straight continuation of the tibial tunnel results in meeting the intercondylar notch at about 11 o'clock, upsetting the native isometric construction. Most known devices disregard this disadvantage and drill through the tibial tunnel. This may account for the impaired rotational stability and long term degenerative changes observed in some cases following traditional ACL reconstruction.

As early as the 1980's the importance of finding the correct origin and insertion points has been recognized. By example, U.S. Pat. No. 4,883,048 discloses a drill guiding apparatus for finding and targeting those points and for guiding drilling tools to the targeted positions. The tibial aiming and drilling is performed in the conventional manner; however the femoral aiming and drilling are independently performed from a posterior-lateral portal from the outside into the joint space, making correct aiming to a distant point difficult.

A different approach is to drill the femoral tunnel inside-out, using flexible guide wires and flexible reamers. This approach is realized for example in the Smith&Nephew Acufex Anatomic ACL Guide System. With this instrumentation the femoral tunnel's position is located by a drill guide bent at the distal end, and is introduced into the intercondylar notch from an antero-medial portal. At this stage, for correct orientation of the femoral tunnel the knee must be flexed to 115° to 120°. A flexible guide wire is introduced through the drill guide and is driven into the femur. Flexible reamers are used to form the femoral tunnel by drilling over the wire from the same portal, independently from the tibial tunnel.

### OBJECTS AND BRIEF SUMMARY OF THE INVENTION

An object of the present invention is to provide a surgical implement particularly useful in many surgical procedures involving the manipulation of a wire or like article in a body cavity, and especially in knee reconstruction procedures of the foregoing type wherein the femoral tunnel is drilled according to an inside-out technique using a flexible guide-wire for locating and drilling the tunnel. Another object of the invention is to provide a novel arthroscopic reconstruction surgical procedure of a knee joint having advantages in the existing procedures briefly discussed above.

According to a broad aspect of the present invention, there is provided a surgical implement for use in a surgical procedure involving the manipulation of a wire or like article in a body cavity, the surgical implement comprising: an elongated stem having a longitudinal axis, a proximal end, and a distal end; a handle carried at the proximal end of the elongated stem; a socket carried at the distal end of the elongated stem configured for insertion into a body cavity during the surgical procedure and receiving the wire or like article therein; an actuator carried by the elongated stem and movable from a normal position closing the socket for gripping the wire or like article therein, to an actuated position opening the socket; a finger-piece carried by the actuator adjacent the handle such as to be conveniently engageable by a user, while gripping the handle, to move the actuator from its normal position closing the socket, to its actuated position opening the socket; and a spring urging the actuator to its normal position closing the socket.

Several embodiments of the invention are described below for purposes of example.

In the described embodiments, the socket is in the form of an insert removably mounted to the distal end of the elongated stem by a projection formed in the insert removably receivable within a recess formed in the distal end of the elongated stem. The actuator is movable axially with respect to the elongated stem and has a distal end closing the socket in the normal, spring-urged position of the actuator, and opening the socket in the actuated position of the actuator.

A further embodiment is described wherein the implement further includes a releasable latch for selectively latching the actuator in its normal position closing the socket or in at least one actuated position opening the socket.

In the latter described embodiment, the at least one actuated position includes a partially-open position sufficiently opening the socket to permit it to receive the wire or like article, and a fully open position sufficiently opening the socket to permit it to be removed and replaced by another socket.

As indicated above, such a surgical implement is particularly useful when the surgical procedure is a knee reconstruction procedure, and the socket is configured to enter through the interior-medial portal of the knee into the intercondylar notch of the femur bone for forming the femoral tunnel therein. In this described embodiment, the wire or like article is a guide wire formed with a sharp trocar tip; and the socket is configured to enter the anterior medial portal of the knee into the intercondylar notch of the femur bone to locate a drill, when gripping the guide wire, for forming the femoral tunnel in the femur bone.

According to another aspect of the present invention, there is provided, a method of performing an arthroscopic reconstruction surgical procedure of a knee joint by replacing a cruciate ligament in the knee joint space connecting the femur bone to the tibia bone, the method comprising:

drilling a tibial tunnel through the tibia bone in the required precise location and direction;

introducing a flexible guide wire through the tibial tunnel into the joint space with a distal end of the guide wire formed with a trocar tip projecting into the joint space;

manipulating the trocar tip of the distal end of the guide wire to engage the inner surface of the femoral condyle at the proper location and direction of the femoral tunnel;

rotating the wire to cause the trocar tip to drill the femoral tunnel through the femur bone;

using a cannulated reamer to drill the femoral tunnel over the guide wire;

introducing a replacement cruciate ligament through said tibial and femoral tunnels;

and anchoring the opposite ends of said cruciate ligament.

Further features and advantages of the invention will be apparent from the description below.

### BRIEF DESCRIPTION OF THE DRAWINGS

The invention is herein described, by way of example only, with reference to the accompanying drawings, wherein

FIG. 1 is an anterior view of the right knee in 90° flexion, showing the cruciate ligaments;

FIGS. 2a and 2b are two views of a prior art drill guide, e.g. as described in U.S. Pat. No. 4,883,048, illustrating its use in drilling the tibial tunnel in a knee reconstruction surgical procedure;

FIG. 3 illustrates a bent femoral guide, in the form of a drill guiding wire, for use in drilling the femoral tunnel using a prior art guide system (the Acufex Anatomic ACL Guide System);

FIG. 4 is a flow chart illustrating the novel method of the present invention;

FIG. 5 illustrates a surgical implement constructed in accordance with the present invention for use in a surgical procedure involving the manipulation of a wire or like article in a body cavity and particularly useful in the surgical procedure of FIG. 4;

FIG. 6 illustrates the flexible guide wire, in the procedure illustrated in FIG. 5, inserted into the tibial tunnel and driven into the femur for drilling the femoral tunnel;

FIG. 7 is a longitudinal sectional view illustrating the surgical implement of FIG. 5;

FIG. 8 illustrates the construction of the socket at the end of the elongated stem in the implement of FIG. 7;

FIG. 9 illustrates the closed position of the socket at the distal end of the elongated stem in the implement of FIG. 7;

FIG. 10 is a sectional view illustrating the oblique axis of the bore gripping the guide-wire in the socket of FIG. 9;

FIG. 11 is a view more particularly illustrating the trocar tip of the guide wire gripped by the surgical implement of FIGS. 7-10;

FIGS. 12 and 13 schematically illustrate the open and closed positions of the socket in a modified construction of the surgical implement of the present invention;

FIGS. 14 and 15 illustrate the open and closed positions of another socket construction;

FIG. 16 is an exploded view illustrating another surgical implement constructed in accordance with the present invention;

FIG. 17a is a fragmentary view of the latch in the surgical implement of FIG. 16, as schematically shown in FIG. 17b, when the actuator is in its normal position closing the socket, as shown in FIG. 17c;

FIGS. 18a-18c are corresponding views when the latch is in a partially-open position, as shown in FIG. 18c, for receiving the wire or like article; and

and FIGS. 19a-19c are corresponding views wherein the latch is in its fully-open position wherein the socket, shown in FIG. 19c, is sufficiently opened to permit the socket to be removed and be replaced by another socket.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

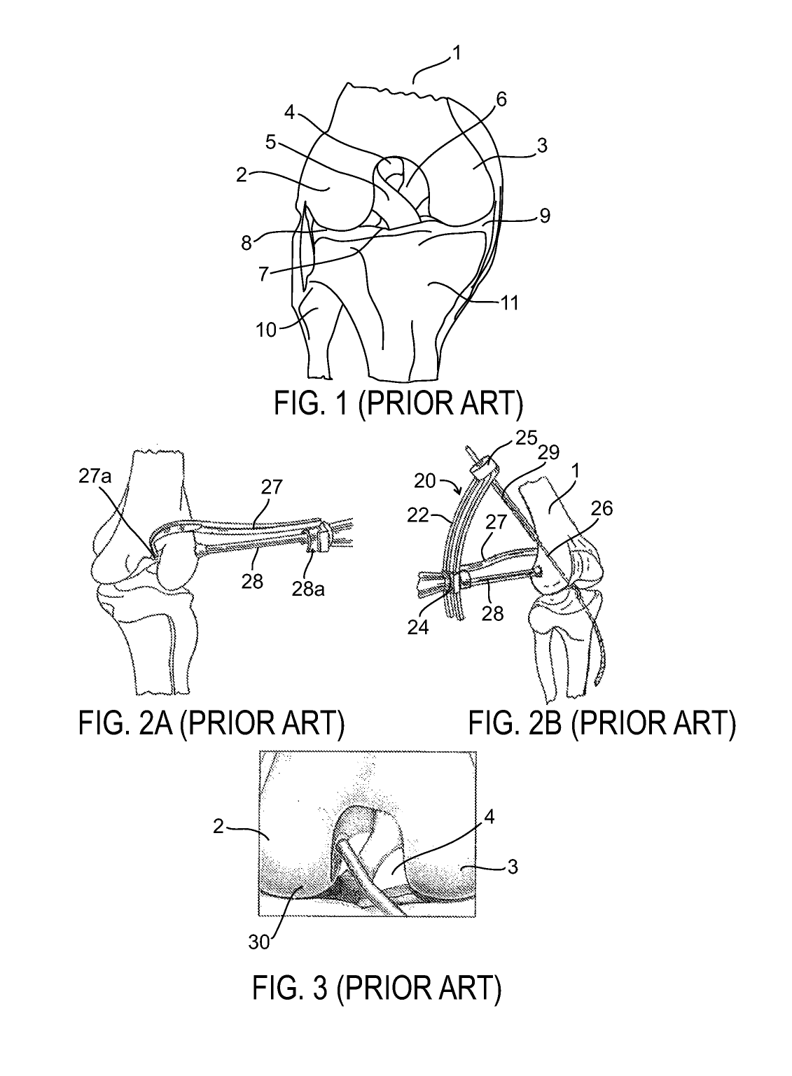

### BRIEF DESCRIPTION OF THE PRIOR ART (FIGS. 1-3)

FIG. 1 is an anterior view of the right knee in 90° flexion, with the kneecap removed. The lateral condyle 2 and the medial condyle 3 of the femoral head 1 are separated by the intercondylar notch 4. The anterior 5 and the posterior 6 cruciate ligaments are shown in their native position between the femoral condyles and the tibial plateau 7. Lateral 8 and medial 9 menisci are shown, as well as the attachment of the fibula 10 to the tibia 11.

FIGS. 2a and 2b show the prior art drill guide which may be used for locating and drilling the tibial tunnel, as described in the previously-mentioned U.S. Pat. No. 4,883,048 to Purnell et al., whereas FIG. 3 shows the previously-mentioned prior art Acufex bent guide which may be used and manipulated for drilling a femoral tunnel after the stump of the torn anterior Cruciate ligament has been surgically removed.

As indicated earlier, U.S. Pat. No. 4,883,048 discloses a drill guide for locating the correct origin and insertion points of the tibial and femoral tunnels and for guiding the drill to produce such tunnels in accordance with the prior art. The drill guide is generally designated 20 in FIG. 2b, and includes an arcuate track 22 presettably mounting a clamping assembly 24 and a guiding assembly 25. FIGS. 2a and 2b illustrate the drill guide 20 being used for drilling the femoral tunnel, schematically indicated at 26, through the lateral condyle 2 of the femoral head 1. For this purpose, the clamping assembly 24 includes a hook member 27 having a hook 27a at its outer end engageable with one side of the lateral condyle 2, and a clamping rod 28 formed at its outer tip with an enlarged head 28a engageable with the other side of the lateral condyle 2. Drill guide assembly 25 includes a guide pin 29 which, when assembly 25 is properly preset on tracks 22 to locate the tip 28a of hook 28 at the target region, aligns the pin with the target region in order to use the pin for guiding a hollow drill to produce the femoral tunnel 26.

Further details of the construction of the drill guide 20 illustrated in FIGS. 2a and 2b, and the manner of using it for drilling both the femoral tunnel 26 through the femur 1, and also the tibial tunnel through the tibia 11, are available in the specification of the above-cited patent which is incorporated herein by reference.

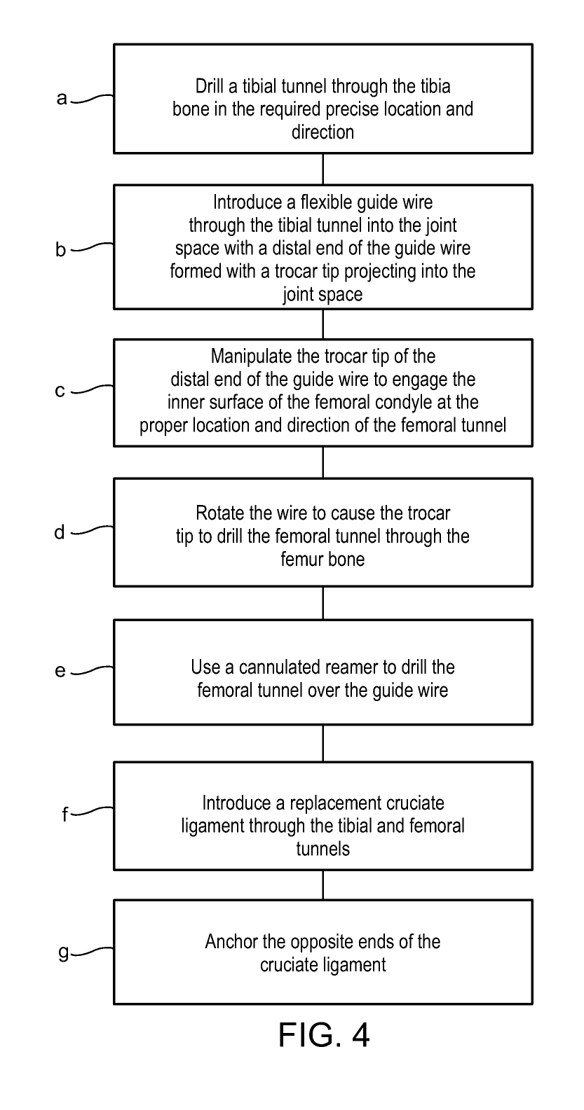

FIG. 3 illustrates the manner of drilling the femoral tunnel inside-out, using flexible guide wires and the above-mentioned Acufex Anatomic ACL Guide System of Smith & Nephew. As mentioned earlier, this technique uses a guiding wire, generally designated 30 in FIG. 4, as a drill guide bent at its distal end, and introduced into the intercondylar notch of the femur bone.

As described in the remaining figures of the drawings, the present invention provides a surgical implement particularly useful for manipulating the guide wire 30 of FIG. 4 in order to affix it to the proper position for using it to drill the femoral tunnel inside-out.

### DESCRIPTION OF PREFERRED EMBODIMENTS OF THE INVENTION

### The Novel Method

As indicated earlier, FIG. 4 is a flow chart illustrating the novel method of the present invention. Thus, as shown in FIG. 4, the novel method involves the following steps:

(a) drilling a tibial tunnel through the tibia bone in the required precise location and direction;

(b) introducing a flexible guide wire through the tibial tunnel into the joint space with a distal end of the guide wire formed with a trocar tip projecting into the joint space;

(c) manipulating the trocar tip of the distal end of the guide wire to engage the inner surface of the femoral condyle at the proper location and direction of the femoral tunnel;

(d) rotating the wire to cause the trocar tip to drill the femoral tunnel through the femur bone;

(e) using a cannulated reamer to drill the femoral tunnel over the guide wire;

(f) introducing a replacement cruciate ligament through the tibial and femoral tunnels; and

(g) anchoring the opposite ends of the cruciate ligament.

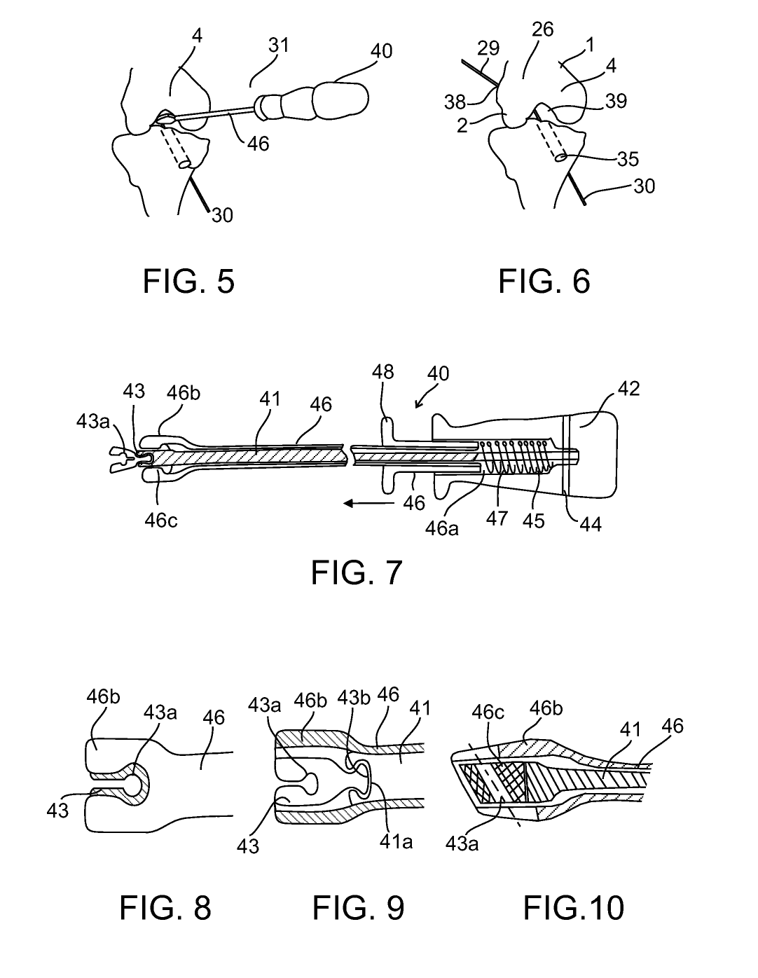

### The Novel Surgical Implement of FIGS. 5-19c

As indicated above, FIG. 5 illustrates a surgical implement, generally designated 40, for manipulating a wire or like article in a surgical procedure, such as in step (c) of the inside-out technique for drilling a femoral tunnel in a knee-reconstruction procedure as described above. As shown therein, the novel surgical implement 40 includes a socket configured to enter through a lateral-medial portal of the knee into the intercondylar notch 4, and to locate a drill bit (not shown) enclosing the guide-wire 30 (FIGS. 3 and 6) in order to drill the femoral tunnel 26 at the exact proper location through the lateral condyle of the femoral head 1 after the tibial tunnel 26a has been drilled through the tibia 11.

As shown particularly in FIG. 7, surgical implement 40 includes an elongated stem 41 carrying at one end (the proximal end) a handle 42, and at the opposite end (the distal end) a socket 43 for receiving the guide wire 30 in FIGS. 4 and 6. The proximal end of stem 41 is fixed within handle 42 by a cross-pin 44. Socket 43 at the distal end of elongated end of stem 41 is in the form of a removable clamping insert, normally in an open position as shown by broken lines 43a in FIG. 7, but clampable to a closed position as shown in full lines in FIG. 7.

Handle 42 is formed, at its distal end, with a cylindrical cavity 45, which receives the proximal end of an actuator, in the form of a sleeve 46. Cavity 45 further includes a spring 47 bearing against the proximal end 46a of actuator sleeve 46, such that the distal end 46b of the actuator sleeve is forced to engage, and close, the elastic clamping element 43 at the distal end of the elongated stem 41.

It will thus be seen that the elastic clamping insert 43 at the distal end of the elongated stem 41 is normally in an open position (shown by broken lines 43a, FIG. 7), but is deformable to a closed position (full lines, FIG. 7) by the movement of the actuator sleeve 47 to its normal spring-urged position, as urged by spring 47, wherein the inner surface 46c of the sleeve distal end engages the outer surface of the socket 43 to cam it to a closed position. It will also be seen that actuator sleeve 46 may be manually moved in the proximal direction to an actuated position to enable the elastic clamping element to move to its normal open position.

A finger-piece 48 is carried by actuator sleeve 46 adjacent to handle 42 such as to be conveniently engageable by a user, while gripping the handle, to manually move the actuator sleeve from its normal position closing the socket, to its actuated position opening the socket.

As shown particularly in FIG. 8, the elastic clamping element 43 is formed with a bore 43a which receives the guide wire 30 (FIG. 3) and firmly grips it when the clamping element is in its closed position. As seen in FIG. 10, bore 43a is not formed perpendicularly to the longitudinal axis of elongated stem 41, but rather at an oblique angle thereto (e.g., 30°-60°)to firmly grip the guide wire therein.

As seen in FIG. 9, the elastic clamping insert 43 is removably mounted to the distal end of the elongated stem 41. For this purpose, the proximal end of the elastic clamping insert 43 is formed with an axial projection 43b slidably received within a recess 41a formed transversely in the distal end of the elongated stem 41.

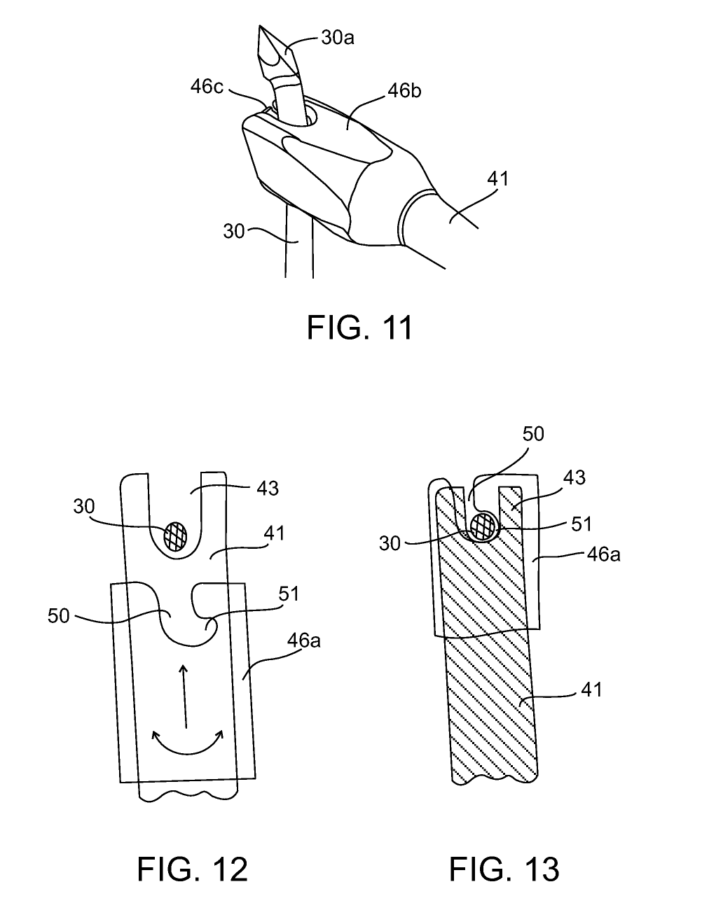

FIG. 11 illustrates the distal end 46b of the actuator sleeve 46, together with the guide wire 30 clamped by the elastic clamping element (43, FIGS. 7-10) within that end of the actuator sleeve. As seen in FIG. 11, the distal end 46b of the actuator sleeve 46 is formed with openings 43c for accommodating the guide wire 30, and is also dimensioned so as to permit the tip of the guide wire 30 to be exposed to the inner surfaces of the bone through which the femur tunnel is to be formed. This exposed tip of guide wire 30 is formed with a sharpened trocar formation 30a such that, when engaging the bone, the guide-wire may be rotated to form a bore in the bone.

FIGS. 12 and 13 illustrate a variation, wherein the distal end 46a of the actuator sleeve 46 is formed with a longitudinally-extending slot 50 terminating in a circumferentially-extending slot 51 to receive the guide-wire 30 and to lock the actuator sleeve 46, together with the elastic clamping element 43 therein, to their closed positions thereof in order to firmly grip the guide-wire 30. Such an operation is produced by first moving the actuator sleeve 46 in the distal direction to pass the guide-wire through slot 50, and then to rotate the actuator sleeve so as to move the guide-wire into slot 51.

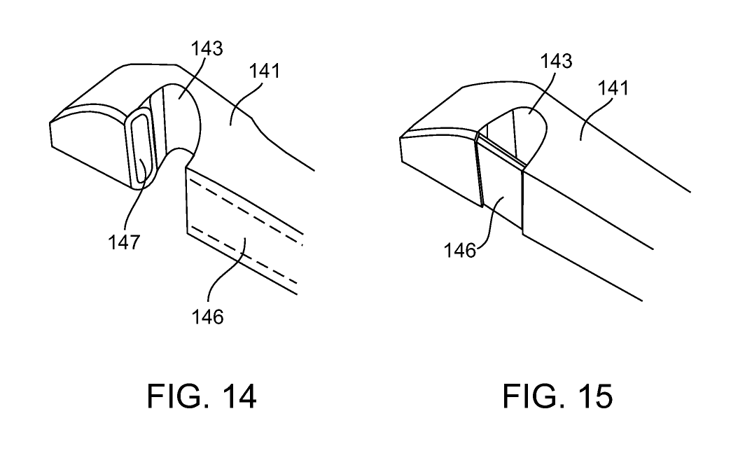

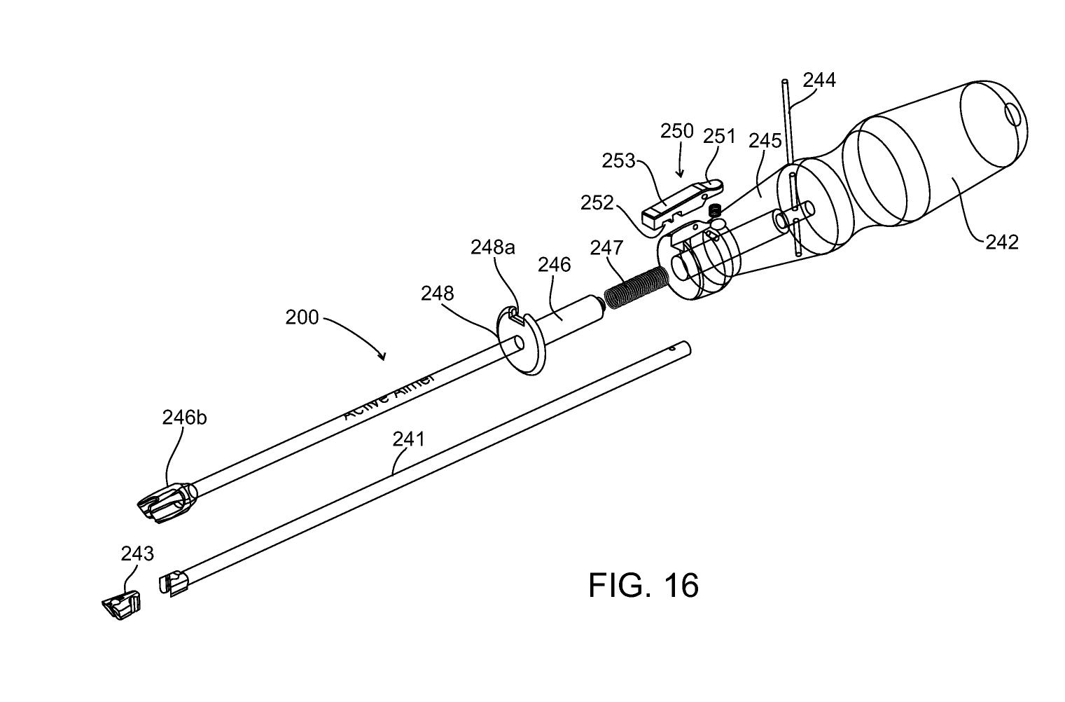

FIGS. 14 and 15 illustrate a further variation in the construction of the distal end of the surgical implement. In this variation, the elongated stem 141 carries, at its distal end, a socket 143 open to one side for receiving the wire (e.g., 30, FIG. 11), and the actuator is in the form of a gate 146 movable to an open position shown in FIG. 15, or to a closed position shown in FIG. 16. In its closed position its distal end is received within a recess 147 (FIG. 14).

The proximal end of the implement illustrated in FIGS. 14 and 15 may be of a similar construction as in FIGS. 5-7, to include a handle (corresponding to 42) for gripping the implement, a cavity (corresponding to 45) for receiving the proximal end of the actuator, a spring (corresponding to spring 47) for biasing the actuator to its normal position, and a finger-piece (corresponding to 48 in FIG. 7) for manually moving the actuator to its actuated position. Such an implement would be particularly useful for receiving the wire or like structure through a lateral opening from the side, rather than through an axial opening from the tip, and could also include a sleeve-type actuator, as in FIGS. 5-7, for opening and closing the socket.

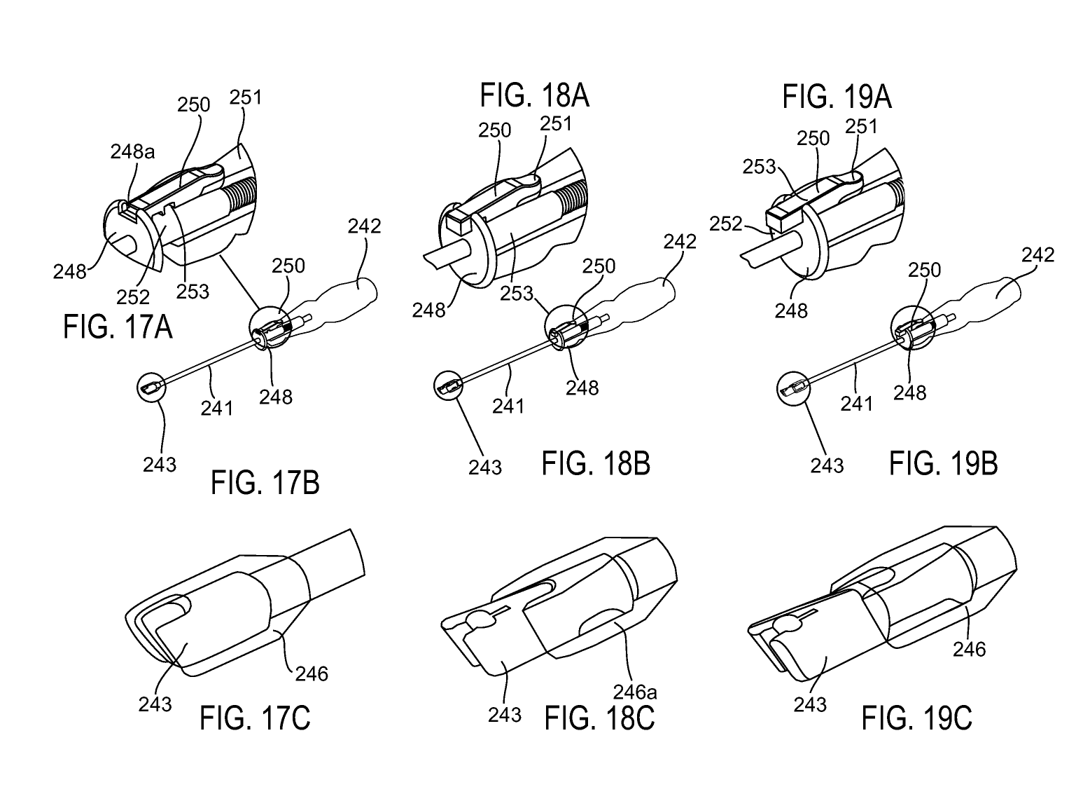

FIG. 16 illustrates another surgical implement constructed in accordance with the present invention, wherein the implement includes a releasable latch for selectively latching the actuator in its normal position closing the socket, or in at least one actuated position opening the socket. FIGS. 17a-19c illustrate the manner in which the latch opens and closes the socket.

Thus, as seen in FIG. 16, the implement therein illustrated, generally designated 200, is of a similar construction as described above with respect to FIG. 7, as including an elongated stem 241 carrying a handle 242 at its proximal end, and a socket 243 at its distal end. The proximal end of elongated stem 241 is fixed to handle 242 by a cross-pin 244. The distal end of the socket is formed with a cavity 245 receiving the distal end of an actuator sleeve 246, and also a spring 247 for biasing the actuator sleeve to its normal extending position wherein its distal end 246b engages the outer surface of socket 243 and cams it to a closed position to grip the wire or other like structure. Actuator sleeve 246 further includes a finger-piece 248, in the form of an annular flange, which may be manually engaged to move the actuator sleeve in the proximal direction, to disengage the distal end 246b of the actuator sleeve from the socket 243, and thereby to permit the socket to move to its normal open position.

Thus, the implement illustrated in FIG. 16 is of basically the same structure, and operates basically in the same manner, as described above with respect to the implement of FIG. 7.

The implement of FIG. 16, however, further includes a latch 250 carried at the distal end of handle 242 for selectively latching the actuator sleeve 246 in its normal position closing the socket, or in at least one actuated position opening the socket. As described below particularly with respect to FIGS. 17a-19c, latch 250 releasably retains the actuator sleeve in one of two open positions, namely a partially-open position as illustrated in FIGS. 18a-18c sufficiently opening the socket to permit it to receive the wire or like article, and a fully-open position, as shown in FIGS. 19a-19c sufficiently opening the socket to permit it to be removed for repair or replacement purposes.

Thus, as shown in FIG. 16 (and also in FIGS. 17a-19a), latch 250 is in the form of a cantilever strip which is fixed at its proximal end 251 to handle 242. At its distal, free end, it includes two latching elements, in the form of grooves 252, 253, cooperable with a notch 248a formed in the finger-piece 248 of actuator sleeve 246.

FIGS. 17a-17c illustrate the position of latch 250 when the actuator is in its normally closed position closing the socket 243 (FIG. 17c). It will be seen that in this normally-closed position of the actuator sleeve, neither latch elements 252, 253 of latch 250 engage slot 248a of finger-piece 248 such as to latch the actuator sleeve, thereby permitting the actuator sleeve to move to its fully closed position under the influence of spring 247.

FIGS. 18a-18c illustrate the partially-open position of latch 250, wherein the outermost latch element or groove 252 engages finger-piece 248 of the actuator sleeve, to thereby latch the actuator sleeve in a partially-open position, as illustrated in FIG. 18c, such as to permit the socket 243 to open to receive the wire or other like article to be gripped by the implement.

FIG. 19a illustrates the latch 250 to its fully-open position, wherein the inner latch element or groove 253 engages finger-piece 248 of the actuator sleeve, to thereby latch the actuator sleeve in its fully open position, as illustrated in FIG. 19c, such as to permit the socket 243 to be removed from the distal end of the implement, and to be repaired, or replaced by another socket as may be required in any particular surgical procedure.

While the invention has been described above with respect to several preferred embodiments, it will be appreciated that these are set forth merely for purposes of example, and that many other variations, modifications and applications of the invention may be made.

What is claimed is:

1. A surgical implement for use in a surgical procedure involving the manipulation of a wire or like article in a body cavity, said surgical implement comprising:
an elongated stem having a longitudinal axis, a proximal end, and a distal end;
a handle carried at the proximal end of said elongated stem;
a socket carried at the distal end of said elongated stem configured for insertion into a body cavity during the surgical procedure and for receiving the wire or like article therein;
an actuator carried by said elongated stem and movable from a normal position closing said socket for gripping the wire or like article therein, to an actuated position opening said socket;
a finger-piece carried by said actuator adjacent said handle such as to be conveniently engageable by a user, while gripping the handle, to move said actuator from its normal position closing said socket, to its actuated position opening said socket;
and a spring urging said actuator to its normal position closing said socket.

2. The surgical implement according to claim 1, wherein said socket is in the form of an insert removably mounted to the distal end of the elongated stem by a projection formed in the insert removably receivable within a recess formed in the distal end of said elongated stem.

3. The surgical implement according to claim 1, wherein said actuator is movable axially with respect to said elongated stem, said actuator having a distal end closing said socket in the normal, spring-urged position of the actuator, and opening said socket in the actuated position of the actuator.

4. The surgical implement according to claim 3, wherein said actuator is in the form of a sleeve, said finger-piece is carried by said sleeve, and said socket includes an elastic clamping element normally in an open position, but deformable to a closed position by the movement of said actuator sleeve to its normal spring-urged position for gripping the wire or like article.

5. The surgical implement according to claim 4, wherein the distal end of said actuator sleeve has an inner surface engageable with the outer surface of said elastic clamping element to deform it to its closed position when the actuator sleeve is moved to its normal spring-urged position.

6. The surgical implement according to claim 5, wherein said elastic clamping element is formed with a bore configured to receive said wire or like article.

7. The surgical implement according to claim 6, wherein said bore has an axis which is oblique to the longitudinal axis of said elongated stem to enable firm gripping of the wire or like article in the closed position of said elastic clamping element.

8. The surgical implement according to claim 6, wherein said elastic clamping element is in the form of an insert removably mounted to the distal end of said elongated stem by a projection in the insert removably received in a recess in the distal end of said elongated stem.

9. The surgical implement according to claim 1, wherein said handle is formed with a cavity at its distal end, and wherein said cavity receives the proximal end of said actuator and said spring, such as to urge said actuator to its normal, spring-urged position closing said socket at the distal end of said elongated stem.

10. The surgical implement according to claim 1, wherein said finger-piece is carried by said actuator at a location thereof spaced from the distal end of said handle in the normal spring-urged position of the actuator.

11. The surgical implement according to claim 1, wherein said actuator is formed at its distal end with a longitudinally-extending slot terminating in a circumferentially-extending slot for receiving the wire or like article when the actuator is moved to its normal spring-urged position, and for locking same therein when the actuator is rotated about the longitudinal axis of the elongated stem.

12. The surgical implement according to claim 1, wherein said actuator includes a gate closing said socket when the actuator is in its normal, spring-urged position, and opening said socket when the actuator is in its actuated position.

13. The surgical implement according to claim 1, wherein said wire or like article is formed with a sharp trocar tip; and wherein said socket at the distal end of the elongated stem is dimensioned to expose said tip to tissue to be cut and to permit the rotation of the wire or like article when gripped by said socket.

14. The surgical implement according to claim 1, wherein said implement further includes a releasable latch for selectively latching said actuator in its normal position closing said socket or in at least one actuated position opening said socket.

15. The surgical implement according to claim 14, wherein said at least one actuated position includes a partially-open position sufficiently opening said socket to permit it to receive said wire or like article, and a fully open position sufficiently opening said socket to permit it to be removed for repair or replacement purposes.

16. The surgical implement according to claim 14, wherein said actuator is in the form of a sleeve, said finger-piece is carried by said sleeve, and said socket includes an elastic clamping element normally in an open position, but deformable to a closed position by the movement of said actuator sleeve to its normal spring-urged position for gripping the wire or like article.

17. The surgical implement according to claim 16, wherein said releasable latch is fixed at one end to said handle, and includes a latching element for each of said actuated positions of the actuator sleeve cooperable with said actuator sleeve to releasably retain the actuator sleeve in the respective actuated position.

18. The surgical implement according to claim 17, wherein said finger-piece carried by said actuator is a flange formed at the proximal end of said actuator sleeve, and said latch includes a latching element cooperable with said flange to releasably retain the actuator sleeve in its respective actuated position.

19. The surgical implement according to claim 1, wherein said surgical procedure is a knee reconstruction procedure, and said socket is configured to enter through a lateral-medial portal of the knee into the intercondylar notch of the femur bone.

20. The surgical implement according to claim 19, wherein said wire or like article is a guide wire formed with a sharp trocar tip; and wherein said socket is configured to enter said lateral medial portal of the knee into the intercondylar notch of the femur bone to locate a flexible cannulated reamer, when enclosing the guide wire, for forming a femoral tunnel in the femur bone.

21. A method of performing an arthroscopic reconstruction surgical procedure of a knee joint by replacing a cruciate ligament in the knee joint space connecting the femur bone to the tibia bone, said method comprising:
(a) drilling a tibial tunnel through the tibia bone in the required precise location and direction;
(b) introducing a flexible guide wire through the tibial tunnel into the joint space with a distal end of the guide wire formed with a trocar tip projecting into the joint space;
(c) manipulating said trocar tip of the distal end of the guide wire to engage the inner surface of the femoral condyle at the proper location and direction of the femoral tunnel;
(d) rotating said wire to cause said trocar tip to drill the femoral tunnel through the femur bone;
(e) using a cannulated reamer to drill the femoral tunnel over the guide wire;
(f) introducing a replacement cruciate ligament through said tibial and femoral tunnels; and
(g) anchoring the opposite ends of said cruciate ligament.

22. The method according to claim 21, wherein the implement of claim 1 is used for manipulating said trocar tip of the distal end of the guide wire to engage the inner surface of the femoral condyle at the precise location and direction of the femoral tunnel.
